(19) United States
(12) Patent Application Publication
(10) Pub. No.: US 2011/0288072 A1
(43) Pub. Date: Nov. 24, 2011

(54) Use of ivabradine for obtaining medicaments intended for the treatment of endothelial dysfunction

(75) Inventors: Vidal Benatar, Saint-Cloud (FR); Guy Lerebours-Pigeonniere, Levallois Perret (FR); Jean-Claude Tardif, Laval (CA); Eric Thorin, Montreal (CA); Eric Rheaume, Montreal (CA)

(73) Assignee: LES LABORATOIRES SERVIER, SURESNES CEDEX (FR)

(21) Appl. No.: 13/136,033

(22) Filed: Jul. 21, 2011

Related U.S. Application Data
(63) Continuation of application No. 12/008,437, filed on Jan. 11, 2008.

(30) Foreign Application Priority Data
Jan. 11, 2007 (FR) 07/00189

(51) Int. Cl. A61K 31/55; A61P 3/10; A61P 25/28; A61P 3/00; A61P 9/10; A61P 9/04; A61P 9/12

(52) U.S. Cl. 514/212.07

(57) ABSTRACT

Use of ivabradine, or 3-{3-[{[(7S)-3,4-dimethoxybicyclo [4.2.0]octa-1,3,5-trien-7-yl]-methyl }-(methyl)-amino]-propyl}-7,8-dimethoxy-1,3,4,5 -tetrahydro-2H-3-benzazepin-2-one, its addition salts with a pharmaceutically acceptable acid and their hydrates, for obtaining a medicament intended for the treatment of endothelial dysfunction.

The present invention relates to the use of ivabradine, or 3-{3-[{[(7S)-3,4-dimethoxybicyclo [4.2.0]octa-1,3,5-trien-7-yl]-methyl}-(methyl)-amino]-propyl}-7,8-dimethoxy-1,3,4,5-tetrahydro-2H-3-benzazepin-2-one, of formula (I):

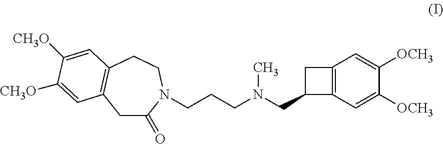

and to addition salts thereof with a pharmaceutically acceptable acid and to hydrates of the said addition salts, for obtaining medicaments intended for the treatment of endothelial dysfunction.

Ivabradine, and also its addition salts with a pharmaceutically acceptable acid, and more especially its hydrochloride, and the hydrates of the said addition salts, have very valuable pharmacological and therapeutic properties, especially negative chronotropic properties (reduction of cardiac frequency), which render those compounds useful in the treatment, prevention and improvement of prognosis of various cardiovascular diseases associated with myocardial ischaemia, such as angina pectoris, myocardial infarction and associated rhythm disorders, as well as in various pathologies involving rhythm disorders, especially supra-ventricular rhythm disorders, and in chronic heart failure.

The preparation and therapeutic use of ivabradine and its addition salts with a pharmaceutically acceptable acid, and more especially its hydrochloride, have been described in European Patent EP 0 534 859.

The Applicant has now discovered that ivabradine and its addition salts, more especially its hydrochloride, have valuable properties that allow them to be used in the treatment of endothelial dysfunction.

Under normal physiological conditions, the endothelium forms a semi-permeable layer between the circulating elements of the blood and the wall of all blood vessels, whether venous or arterial. Although formed by a single layer of cells, its total volume is comparable with that of the liver (Huttner and Gabbiani, Vascular endothelium in hypertension. Hypertension, ed. Genest J. Kuchel O, Hamet P and Cantin M. New York: McGraw-Hill, 1983, p. 473-488) and its activity is very diversified. In response to numerous substances (circulating hormones, cytokines, medicaments), to physical or chemical stimuli (shearing force, change in pressure, pH), the endothelial cells synthesise and release various factors that modulate angiogenesis, inflammatory responses, haemostasis, vascular tonus, the synthesis and degradation of the extracellular matrix and vascular permeability (Félétou and Vanhoutte, Am J Physiol Heart Circ Physiol 2006, 291:985-1002).

One of the principal protective substances synthesised by the endothelium is nitrogen monoxide or nitric oxide (NO). NO relaxes the smooth muscle cells and inhibits platelet aggregation.

Endothelial dysfunction is the alteration of normal function of the endothelial cells. It is characterised by a progressive incapacity of the vessels to adapt to the environment and to respond to physiological stimuli.

The first works identifying endothelial dysfunction date from the 1980s. A decrease was observed in endothelium-dependent relaxation measured in the aorta of the hypertensive rat (Lockette et al., Hypertension 1986, 8:1161-1166) or in the hypercholesterolaemic rabbit (Verbeuren et al., Circ Res 1986, 58:552-564). Similar observations were then reported on the coronary arteries of atherosclerotic patients, suggesting that endothelial dysfunction might be an early indicator of atherosclerosis (Ludmer et al., N Engl. J Med 1986, 315:1046-1051).

Today, it is associated not only with hypertension or atherosclerosis but also with other physiological and physiopathological processes, such as age, heart or kidney failure, coronary syndrome, type I and type II diabetes, obesity, erectile dysfunction, inflammation, thrombosis, sepsis . . . (Félétou and Vanhoutte, Am J Physiol Heart Circ Physiol 2006, 291:985-1002).

Although endothelial dysfunction is described in a wide variety of pathologies, a common denominator has been identified: oxidative stress. Free radicals play a central role in vascular physiology and physiopathology; they are capable especially of inhibiting the three major pathways of endothelium-dependent vasodilation (nitrogen monoxide, prostacyclin and endothelium-dependent hyperpolarising factor).

The Applicant has now found that ivabradine is capable of restoring endothelial function in coronary, renal and cerebral arteries in animals in which endothelial function has been altered.

That effect allows the use of ivabradine, its addition salts with a pharmaceutically acceptable acid and their hydrates to be considered in the treatment of endothelial dysfunction, especially in patients with heart failure, dyslipidaemia, diabetes or hypertension or suffering from metabolic syndrome, and also in the prevention of, the slowing down of and the treatment of coronary atherosclerosis and its cardiac complications, cerebral atherosclerosis and its ischaemic and thrombotic complications, and atherosclerosis at all levels of the arterial tree.

In addition, the beneficial effect of ivabradine on the cerebral artery (prevention of endothelial dysfunction and improvement of vascular compliance) allows the use of ivabradine in neuroprotection to be considered. Indeed, endothelial dysfunction is associated with cerebral ischaemia and with Alzheimer's disease (Cippola et al., Stroke 2000; 31:940-945; Elesber et al., Neurobiology of Aging 2006; 27:446-450).

The invention thus relates to the use of ivabradine, its addition salts with a pharmaceutically acceptable acid and their hydrates, for obtaining pharmaceutical compositions intended for the treatment of endothelial dysfunction to prevent vascular, cardiac and cerebral complications thereof

The pharmaceutical compositions shall be presented in forms suitable for administration by the oral, parenteral, transcutaneous, nasal, rectal or perlingual route, and especially in the form of injectable preparations, tablets, sublingual tablets, glossettes, gelatin capsules, capsules, lozenges, suppositories, creams, ointments, dermal gels.

Amongst the pharmaceutical compositions according to the invention there may mentioned more especially those which are suitable for oral, parenteral or nasal administration, tablets or dragées, sublingual tablets, gelatin capsules, lozenges, suppositories, creams, ointments, dermal gels and also pharmaceutical compositions with a controlled, slow, prolonged or delayed release.

Apart from ivabradine, one of its addition salts with a pharmaceutically acceptable acid, or one of the hydrates of ivabradine or one of its addition salts, the pharmaceutical compositions according to the invention contain one or more excipients or carriers, such as diluents, lubricants, binders, disintegrants, absorbents, colourants or sweeteners.

By way of example, and without implying any limitation, there may be mentioned:

as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone (PVP),
as disintegrants: agar, alginic acid and its sodium salt, effervescent mixtures.

The useful dosage varies according to the sex, age and weight of the patient, the administration route, the nature of the disorder and of any associated treatments, and ranges from 1 to 500 mg of ivabradine per 24 hours and more especially from 10 to 15 mg per day, and even more especially from 5 to 15 mg per day.

### BRIEF DESCRIPTION OF THE DRAWINGS

FIGS. 1A and 1B show the prevention by ivabradine hydrochloride (IVA) of endothelial dysfunction in the rat suffering from heart failure (HF).

FIGS. 1C and 1D show the prevention by ivabradine hydrochloride (IVA) of endothelial dysfunction in the dyslipidaemic mouse (DL).

### EXAMPLE 1:

### Pharmacological Study

### Effect of Ivabradine Hydrochloride on Endothelial Dysfunction in the Dyslipidaemic Mouse and in the Rat with Heart Failure

For the purpose of simplification, “ivabradine hydrochloride” has been replaced by “ivabradine” in Example 1 below.

The impact of treatment with ivabradine on endothelial function was studied in two pathological animal models, dyslipidaemic mice, and rats with heart failure, on 4 different vascular beds, renal and cerebral arteries in the mouse, coronary and mesenteric arteries in the rats. Study of the endothelial function comprises measuring the capacity of the vessel to dilate in response to various stimuli, such as acetylcholine, or measuring the increase in flow in the vessel.

Some animals were treated with ivabradine, 10 mg/kg per day for 3 months. Dyslipidaemic mice, rats with heart failure, healthy mice (Wild type) and healthy rats, untreated, acted as controls. At the end of the treatment period, the arteries were isolated and mounted in a myograph in order to measure their diameter after the application of acetylcholine or to measure the increase in flow. In order to explore more closely the mechanism associated with endothelial dysfunction, inhibitors of the principal pathways of vasodilation were tested: a free-radical chelating agent, a nitrogen monoxide production inhibitor, a prostacyclin pathway inhibitor.

The compliance (capacity of the vessel to dilate as the transmural pressure increases) of the cerebral arteries of the mice was also evaluated.

### Results

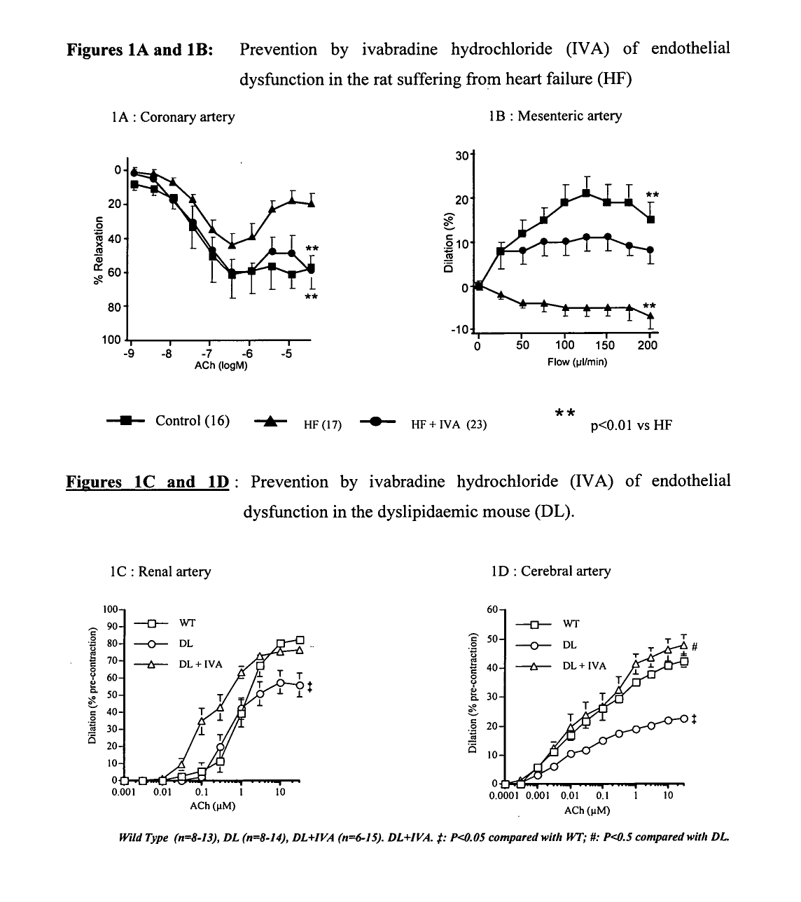

In the rats with heart failure (HF) and the dyslipidaemic mice (DL), endothelial dysfunction was observed in all the vessels studied. Compared with the healthy animals, the capacity of those vessels to dilate is decreased by approximately 20% in the coronary, renal and cerebral artery, and it is zero in the mesenteric artery of the HF rat (FIG. 1).

After 3 months' treatment, ivabradine induces a significant reduction (10-20%) in cardiac frequency. Ivabradine completely prevents entothelial dysfunction in the coronary, renal and cerebral arteries. The dilation measured in those arteries is similar to that measured in the arteries from the healthy animals (FIG. 1A, C, D). The capacity of the mesenteric artery of the treated HF rat to dilate (FIG. 1B) is appreciably improved compared with the artery from the HF rat.

The mechanisms involved in those beneficial effects of ivabradine are a decrease in oxidative stress and preservation of the nitrogen monoxide pathway.

Finally, the compliance of the isolated cerebral arteries of treated mice is improved.

### EXAMPLE 2:

### Pharmaceutical Composition

Formulation for the preparation of 1000 tablets each containing a dose of 5 mg of ivabradine base:

| | | |
|---|---|---|
| Ivabradine hydrochloride | 5.39 | g |
| Maize starch | 20 | g |
| Anhydrous colloidal silica | 0.2 | g |
| Mannitol | 63.91 | g |
| PVP | 10 | g |
| Magnesium stearate | 0.5 | g |

1. A method of treating endothelial dysfunction or a condition associated with endothelial dysfunction in a subject in need thereof comprising administering an effective amount of ivabradine, or 3-{3-[{[(7S)-3,4-dimethoxybicyclo[4.2.0]octa-1,3,5-trien-7-yl]-methyl}-(methyl)-amino]-propyl}-7,8-dimethoxy-1,3,4,5-tetrahydro-2H-3-benzazepin-2-one, or an addition salt thereof with a pharmaceutically acceptable acid, alone or in combination with one or more pharmaceutically acceptable excipients, to the subject.

2. The method of claim 1, wherein the subject is afflicted with heart failure, dyslipidaemia, diabetes, hypertension or metabolic syndrome.

3. The method of claim 1, wherein the condition associated with endothelial dysfunction is atherosclerosis.

4. The method of claim 1, wherein the treatment results in neuroprotection.

5. The method of claim 1, wherein the condition associated with endothelial dysfunction is cerebral ischaemia or Alzheimer's disease.
